(19) United States
(12) Patent Application Publication
(10) Pub. No.: US 2003/0236481 A1
(43) Pub. Date: Dec. 25, 2003

(54) Hemofiltration filter with high membrane utilization effectiveness

(76) Inventor: Jeffrey Burbank, Boxford, MA (US)

Correspondence Address: PROSKAUER ROSE LLP, PATENT DEPARTMENT, 1585 BROADWAY, NEW YORK, NY, 10036-8299, US

(21) Appl. No.: 10/337,948

(22) Filed: Jan. 6, 2003

Related U.S. Application Data
(60) Provisional application No. 60/346,458, filed on Jan. 7, 2002.

(51) Int. Cl. A61M 37/00; B01D 11/00

(52) U.S. Cl. 604/5.01; 604/6.11; 210/646

(57) ABSTRACT

The costs of some blood treatments are strongly driven by the cost of the filter media. For example, hemofiltration and hemodiafiltration filters use expensive media to process blood. In operation, most of the pressure drop occurs near the input end of the filter. Since pressure is what drives fluid across the filter, this results in a low utilization of the filter media toward the outlet end. Also, as blood runs across the media, it lays down blankets of oriented proteins, which occlude flow through, and across, the media. According to the invention, a short filter with a flow restriction at the outlet maintains high blood-filtrate pressure differential and concomitant high utilization. Also, by reversing the flow of blood periodically, the occluding material may be removed increasing utilization overall and permitting the use of smaller quantities of expensive media.

### RELATED APPLICATION

This application is based upon provisional application Ser. No. 60/346,458, entitled “HEMOFILTRATION FILTER WITH HIGH MEMBRANE UTILIZATION EFFECTIVENESS,” filed on Jan. 7, 2002 for Jeffrey H. Burbank and James M. Brugger. The contents of this provisional application are fully incorporated herein by reference.

### BACKGROUND OF THE INVENTION

1. Field of the Invention

The invention relates to membranes and more particularly to membranes used for hemofiltration and hemodiafiltration in which blood is subjected to a more uniform high pressure over the entire membrane to achieve a higher membrane utilization factor.

2. Background

Hemofiltration and hemodiafiltration employ various types of superfine membrane media or filter media. (As used in the specification, the terms “membrane” and “filter” are used interchangeably, irrespective of whether transport is governed by osmosis or convection). One type of membrane media has a tubular structure and is used, for example, by circulating blood through the inside and retrieving waste fluid or circulating dialysate on the outside. Generally a large number of such tubular media elements are connected in parallel through supply and return headers. A jacket surrounding the parallel bundle of tubular media contains the circulating dialysate, waste fluid, or other fluid. In use, blood is pumped at considerable pressure through the blood side of the filter and suffers significant change in pressure as it passes through the narrow tubular media. The narrow passages of the tubular media can become even more occluded with time restricting filtration and removal of waste and causing even greater pressure change. Such changes in pressure are associated with the tearing and breaking of blood cells (hemolysis), which is undesirable.

In addition, the utilization factor of filters is diminished due to the fact substantial pressure drop through the filter. Because of the pressure-drop, most of the fluid extraction occurs at the input end of the filter where the trans-membrane pressure (TMP) is high and much less (or none) at the downstream end. The TMP at the downstream end may be very low because of pressure loss along the restrictive blood path through the media. Since in hemofiltration and hemodiafiltration fluid removal is an important part of the process, the unutilized media may represent a significant fraction of the total and is undesireable and wasteful of the expensive media material. Although tubular media are the most common ones used in this context, other types of media, such as planar media, can also suffer the same problem where the blood path is restrictive.

There exists an on-going need in the art to increase the performance of blood filters and membranes such as used in dialysis and hemofiltration. This is true even in the absence of the clogging effect described above. Also, there exists a need for the pressure changes suffered by blood in extracorporeal blood circuits to be minimized.

### SUMMARY OF THE INVENTION

The costs of many blood treatment processes that involve the use of filters are strongly driven by the cost of the media. For example, hemofiltration filters usually employ very expensive media. A particular type, in common use, is tubular media filters, which are designed with a fairly long body with long media tubes. As a result of the elongated narrow path, the pressure drop through these filters can be high. In hemofiltration and hemodiafiltration, the points at which blood is at a high pressure—i.e., the upstream points—are the points where most the TMP is highest and consequently the points where most of the fluid removal occurs. As a result, the downstream end of the filter can end up serving little purpose, in hemofiltration terms, beyond a flow restrictor to insure higher TMP at the upstream end where the filter utilization is high.

In operation, blood runs parallel to the surface of this media (filter or membrane). Exacerbating the problem of fall-off of TMP is the accumulation of blood products that can narrow the blood flow path. Characteristically, blood lays down blankets of oriented proteins (and other matter) which occlude flow through the blood path, thereby retarding flow of filtrate through the blood and increasing the pressure-drop effect. This also increases the TMP required to effect a given degree of blood filtration because the flow of filtrate through the media material is impeded.

The problems caused by protein deposition can be reduced in two ways. First, the performance can be increased by providing a flow restriction at the outlet of the blood side of the filter to increase static pressure on the downstream blood side of the filter. This enhances membrane utilitization by increasing the TMP. Also, by varying the direction of strain in the blood flow boundary layer adjacent the media, the oriented layer of proteins may be disrupted and/or prevented from forming. This makes it possible to increase the performance of the filter dramatically by increasing the downstream blood-side static pressure (TMP) and by increasing flow of filtrate across the membrane.

One of the mechanisms for varying the strain of the blood adjacent the media to remove the occluding layer of protein and other blood products is to change the direction of flow periodically. An invention for addressing this problem is described in the commonly assigned copending application entitled “Device and Method for Enhancing Performance of Membranes,” U.S. patent application Ser. No. 60/324,437, which is hereby incorporated by reference as fully set forth in its entirety herein. The inventive strategy is, in an embodiment, to reverse the flow of blood through the filter periodically to remove the protein layer. However, this does not completely eliminate the problem of low utilization of the downstream end of the filter for fluid extraction (called “ultrafiltration”).

The static pressure on the downstream end of the blood side of the filter may be increased by various mechanisms. A flow restriction may be formed by placing a clamp on the blood-side tubing near the filter outlet. Alternatively, a capillary tube at the filter outlet or a molded restriction may be provided. This increases the TMP throughout the filter and enhances membrane utilization.

As a result of the increased utilization factor of the media, the filter may be reduced in size in the flow dimension. One option would provide a filter with a relatively short body with short tubular media so that the pressure drop across it is low. This filter is particularly suited to applications in which a substantial TMP needed, for example, hemofiltration.

By using a flow restriction, instead of expensive tubular media as a de facto flow-restriction mechanism, higher media utilization may be obtained. By combining the flow restriction with the filter flow reversal technique of the patent incorporated by reference above, significant economies can be achieved in the filter, which is the most expensive consumable in hemofiltration, hemodiafiltration systems, and other systems where substantial pressure drop from blood to filtrate side is needed.

The invention will be described in connection with certain preferred embodiments, with reference to the following illustrative figures so that it may be more fully understood. With reference to the figures, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

FIG. 1A is an illustration of a filter according to the prior art along with a TMP profile along the length of the filter.

FIG. 1B is an illustration of a hemofiltration filter according to an embodiment of the invention along with a TMP profile along the length of the filter.

FIG. 1C is an illustration of an alternative design for a hemofiltration filter according to an embodiment of the invention.

FIG. 2A is a diagram of the surface of a piece of filter or membrane with blood flowing past it showing the attachment of proteins and other blood factors to form an occluding layer on the media/membrane.

FIG. 2B is a diagram of the surface of FIG. 1 in which the strain of the blood near the surface is reversed, which may be caused, as shown, by the reversal of the flow of blood, causing a disruption of the layer of proteins and other blood factors.

FIGS. 3A and 3B illustrate a single-layer planar filter medium/membrane and an alternative mechanism for straining the layer of proteins and other factors causing occlusion.

FIG. 4 is an illustration of a multiple planar layer filter media/membrane in which occlusion may occur.

FIG. 5 is an illustration of an extracorporeal blood circuit for reversing the flow of blood through a filter without changing the direction of the flow of blood through the patient.

FIG. 6 is an illustration of the extracorporeal blood circuit for reversing the flow of blood through a filter without changing the direction of the flow of blood through the patient in which replacement fluid is added on a patient side of the blood circuit.

FIG. 7 is an illustration of extracorporeal blood circuit for reversing the flow of blood through a filter without changing the direction of the flow of blood through the patient according to an embodiment of the invention in which replacement fluid is added on a filter side of the blood circuit and a flow direction selector switch is used to determine to which side of the filter replacement fluid is added.

FIGS. 8A and 8B show a type of manifold used for tubular media filters that prevents the settling coagulation of blood by promoting vigorous mixing in the manifold.

FIG. 9 shows a short flow restrictor for increasing trans-membrane pressure in a filter.

FIG. 10 shows an elongated flow restrictor or capillary for increasing trans-membrane pressure in a filter.

### DETAILED DESCRIPTION OF THE PREFERRED EMBODIMENTS

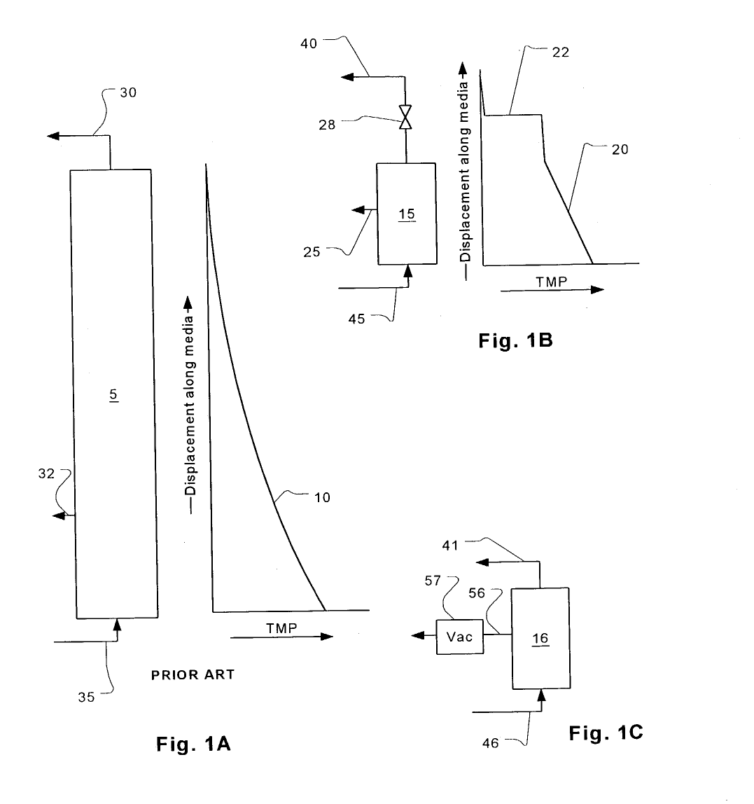

Referring to FIG. 1A, prior art hemofiltration and hemodiafiltration systems employ a filter 5. In the current example, the filter is of a known type having a large number of media tubules. As blood passes through the tubules of the filter 5, whose passages are very fine, the blood is subjected to a significant loss of static pressure (or trans-membrane pressure; TMP) as indicated by the curve 10. Flow friction causes the static pressure to drop and hence the TMP. Also, the accumulation of proteins and other blood constituents on the filter media reduces the flow area and increase frictional losses, particularly at the upstream end where the flow through the filter is greatest.

Because the rate of fluid removal from the blood is driven by the TMP, the utilization of the filter media near the downstream end 30 is low. Graphically this is illustrated by the TMP curve 10, which indicates that the TMP is highest near the upstream end 35. Since TMP drives filtration, the filter utilization is highest where the TMP is highest. Since media utilization is low at the downstream end, the downstream end of the filter 5 is acting primarily as flow restrictor rather than functioning as a fluid filter.

Referring now also to FIG. 1B, another filter 15 with substantially less filter area, but with a much higher utilization factor as can be seen by its TMP profile 20. The higher utilization is derived from two contributing sources. First, a flow throttling device 28 is positioned in a filter outlet line 40. The flow throttling device 28 may be an adjustable valve, an orifice, a restricted-size passage in the outlet line or a capillary. Many such devices are known and the concept need not be expanded on for purposes of understanding the invention. A capillary may provide certain advantages in terms of the amount of pressure drop relative to the potential for flow reversal or degree of turbulence caused by mean flow acceleration/deceleration. By restricting flow at the outlet of the filter 15, the TMP within the filter 15 is high over substantially all of the filter's 15 length. This is because some of the pressure drop occurs across the flow throttling device 25. Note that the filter 15 need not be shorter than a conventional filter, such as 5, to achieve the higher utilization effect.

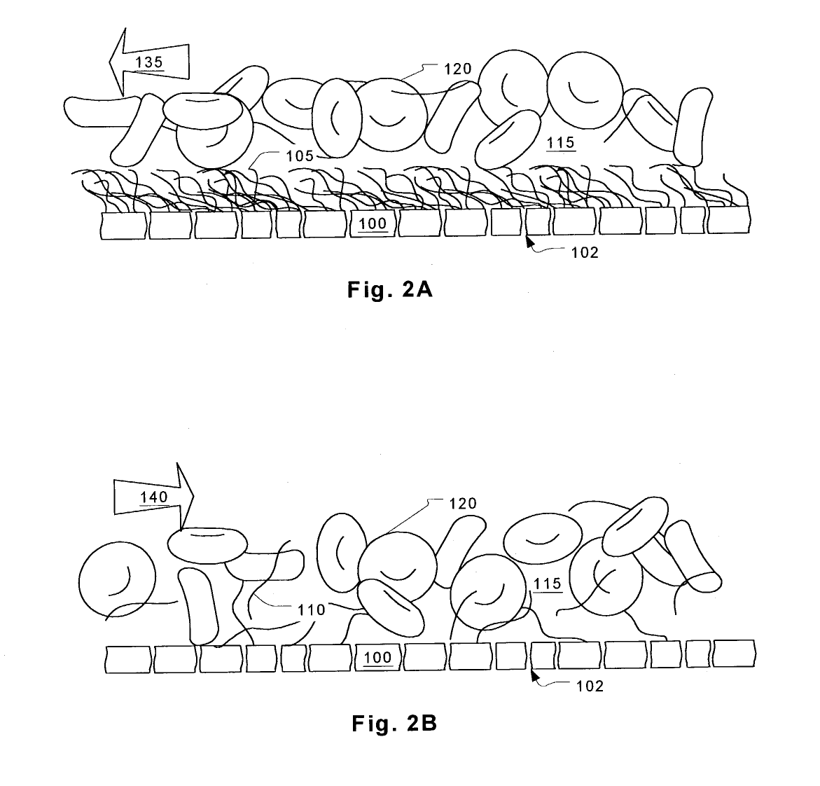

A second component of high utilization of the filter 25 has to do with the blocking of the media by protein and other factors precipitating on the media surface. As mentioned in connection with FIG. 1A, a substantial portion of the pressure drop may occur near the input end 35 of the filter 5 in part because of occlusion by precipitated material on the filter media. Thus, even though the pressure is high, the utilization of media even at the input end 35 may be low. To use a smaller filter, ideally, some means for avoiding this problem may be employed. This issue is discussed below in connection with FIGS. 2A and 2B.

Note that the pressure drop through the flow throttling device 25 is preferably a substantial fraction, if not a majority of that through the filter 15-flow throttling device 25 combination. Note also that the goal is to provide a pressure differential between the blood side and the filtrate side. Referring to FIG. 1C, this may be accomplished also by placing a vacuum on the filtrate side of the filter and, as discussed, a low pressure loss through the blood side so that the pressure differential over the whole media surface is high. Note that the type of media with which the above approach may be used is not limited to tubular media. Planar media and other types of filter media may also be employed.

Referring now to FIG. 2A, in a hemofiltration filter, dialysis membrane, or other similar system, blood 115 with blood cells 120 flows past a filter or membrane 100. Fluids and suspended material and/or solutes (not shown) pass through the filter or membrane 100 through pores 102. Blood 115 flows in the direction indicated by the arrow 135 creating a boundary layer in which fluid is strained in the vicinity of the filter or membrane 100. As a result of continuous operation for a period of time, an oriented layer of proteins 105 and other matter accumulates on the surface of the filter or membrane 100.

The filter or membrane 100 may be the wall of a piece of tubular media or membrane as is commonly used in hemofiltration and dialysis. Alternatively, it may be one many closely spaced layers of planar media. The flow of blood is normally driven by pumping through spaces between the layers or passages and the accumulation of proteins 105 and other matter results in occlusion. It interferes with the flow across the media or membrane 100 and it interferes with the transport of suspended material and/or solutes through the media or membrane 100.

Referring to FIG. 2B, to change the direction of the strain of the blood 115 in the vicinity of the filter or membrane 100, the direction of the flow of blood 115 may be reversed as indicated by the arrow 140. As a result of the change in the strain in the layer near the filter or membrane 100, the protein 105 and other matter 116 that was deposited on the filter or membrane 100 is disrupted and, to some extent, dislodged as indicated at 110. This removes the impediments to flow across the media or membrane 100 and to the transport of suspended material and/or solutes through the media or membrane 100.

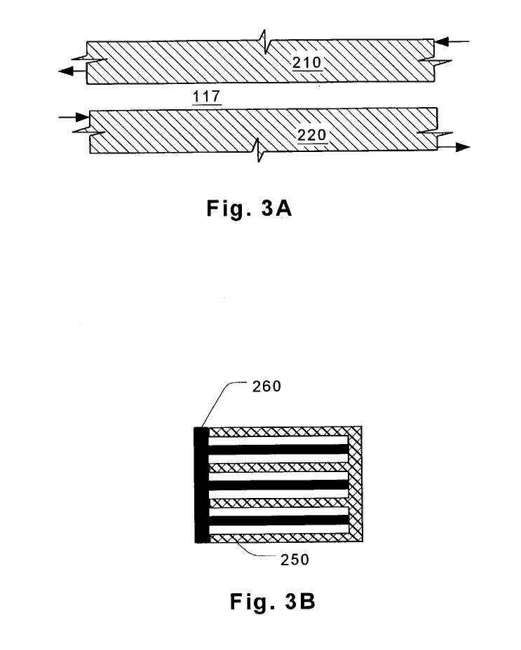

Referring now to FIGS. 3A and 3B, the strain of the blood 117 near the filter or membrane 100 can be reversed, or its direction changed, in ways other than by reversing the flow. For example, as illustrated in FIG. 3A, a planar element 210 opposite a filter or membrane 220 moves relative to the filter or membrane 220 generating a couette flow of blood 117. This effect could be generated in a filter bank of planar filters 250, 260 by stopping the flow and straining the blood by moving every other layer relative to those between them alternatingly in opposite directions.

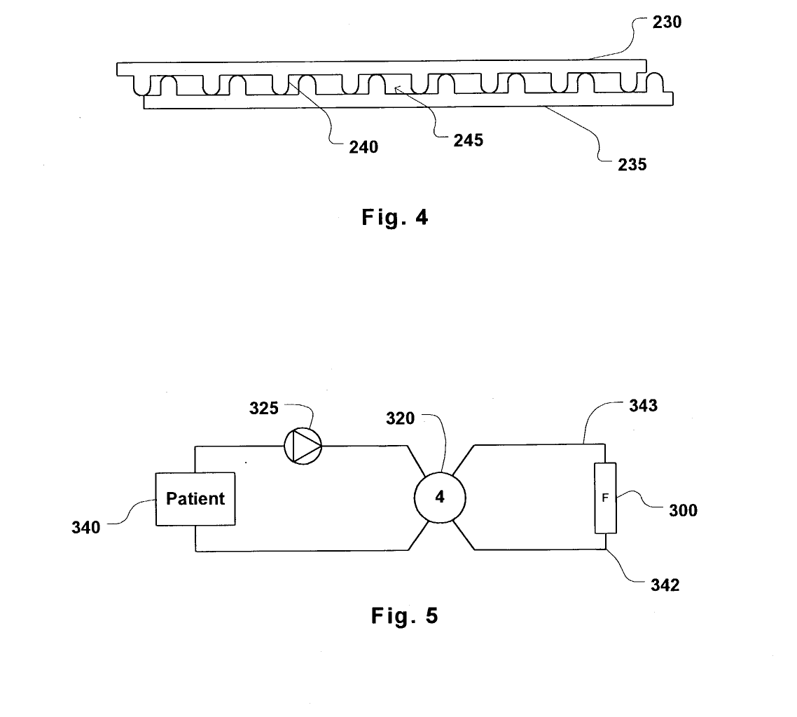

Referring now to FIG. 4, an example of a way to provide closely spaced planar layers of filter or membrane 230, 235 is shown. The adjacent layers of filter or membrane 230, 235 are spaced apart by bumps 240 to create a passages 245 between them. The narrow passages 245 are also susceptible to pressure drop.

Referring now to FIG. 5, an extracorporeal blood circuit draws blood from a patient 340 via a pump 325, runs it through a filter 300 and returns it to the patient 340. In the example embodiment, the circuit includes a four-way valve 320 that switches the blood circuit ends of the filter 342 and 343 such that blood can be run through the filter 300 in either direction selectively depending on the configuration of the four-way valve 320. The pump 325 can run in a single direction and blood is drawn from the patient 340 without changing the draw/return roles of the accesses.

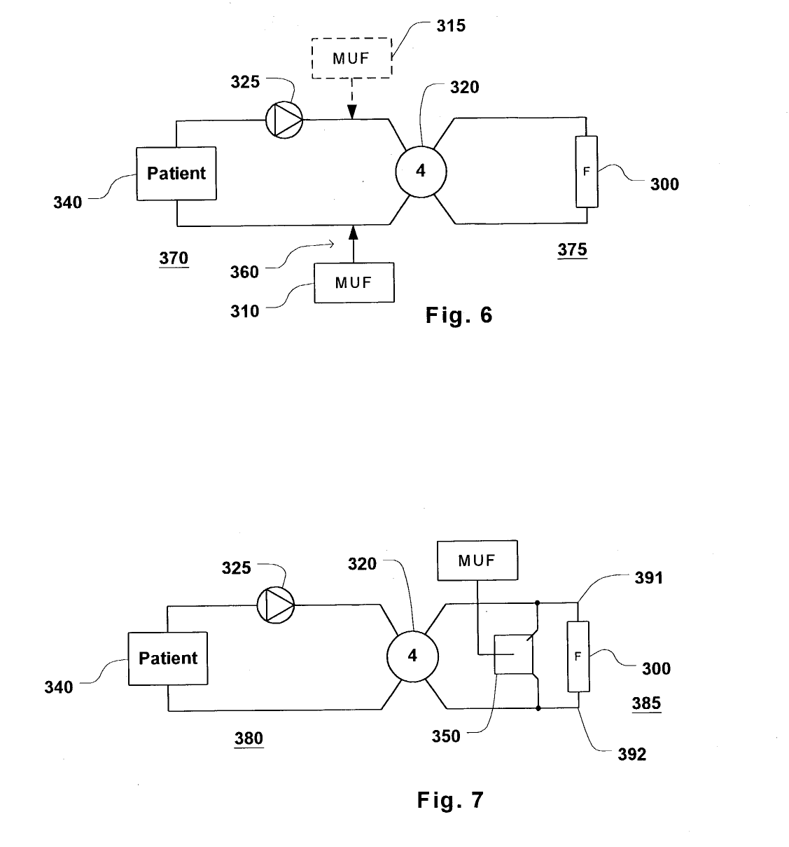

Referring now to FIG. 6, in many applications, replacement fluid must be added to the blood circuit. In the present example, a tap 360 may be added on the patient side 370 of the four-way valve as opposed to the filter side 375. By adding replacement fluid on the patient side of the blood circuit, the replacement fluid is always added at the same point in the filtration process, that is, post-filtration dilution (as illustrated at 320) or pre-filtration dilution (as illustrated at 315). Referring to FIG. 7, in an alternative blood circuit, replacement fluid is added on the filter side 385 of the four-way valve. A flow diverter 350 (in essence, a Y-switch) directs the flow of replacement fluid at a selected one of its to ends 391, 392 according to the current flow direction through the filter 300 and whether the desired effect is pre- or post-dilution. The flow diverter 350 may be of any suitable construction, but is preferably hermetic, similar to the design of the valve design disclosed in the U.S. Patent Application entitled: “Hermetic Valve Permitting Disposable Valve Body” U.S. patent application Ser. No. 09/907,872 is hereby incorporated by reference as if fully set forth herein in its entirety.

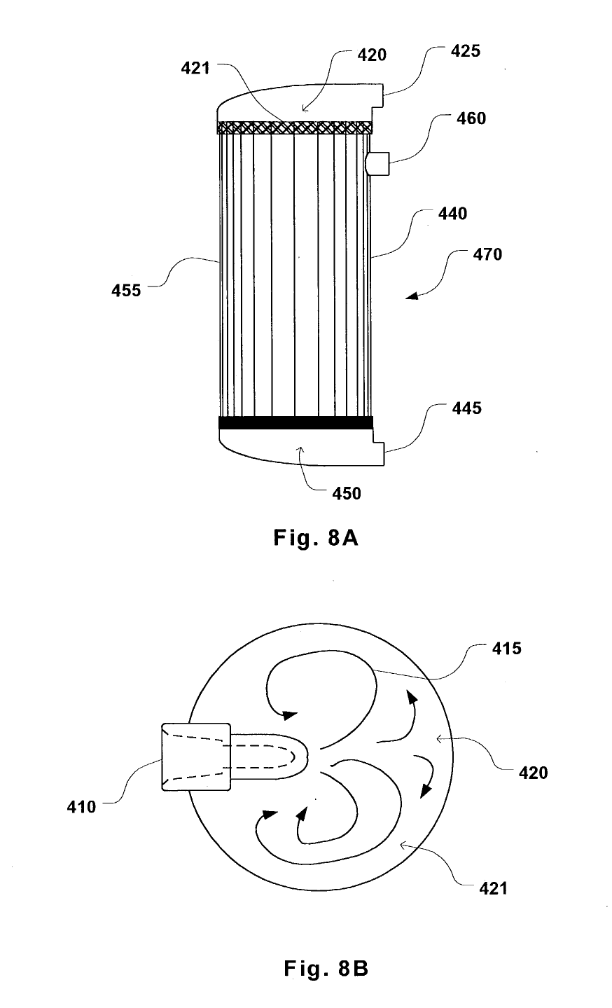

Referring now to FIG. 8A, in tubular media filters such as 470, blood flows into an inlet 425 of an inlet manifold 420 which supplies the flow of blood to multiple media tubules 440 encased in a housing chamber 455. Fluid such as waste fluid or dialysate is circulated or removed through one or more vents 460. As the blood flows through the media tubules 440 fluids are exchanged and/or vented into the housing chamber 455 and the treated blood exits the media tubules 440 into an outlet manifold 450, finally gathering in an outlet 445.

Because the mean flowrate of blood decelerates between the inlet 425 and the inlet manifold 420 and the flowrate in the inlet manifold is slow, there could be a tendency for suspended matter in the blood to settle in stagnant regions of the inlet manifold 420. Referring to FIG. 8B, to prevent this, it is common in the industry to supply the blood into the inlet manifold 420 in a way that generates circulating flow throughout the inlet manifold 420. For example, blood may flow in at a tangent through a centrally located horizontally disposed inlet nozzle 410 creating a jet that produces fast-moving circulating eddy patterns 415 across the surface of the header 421.

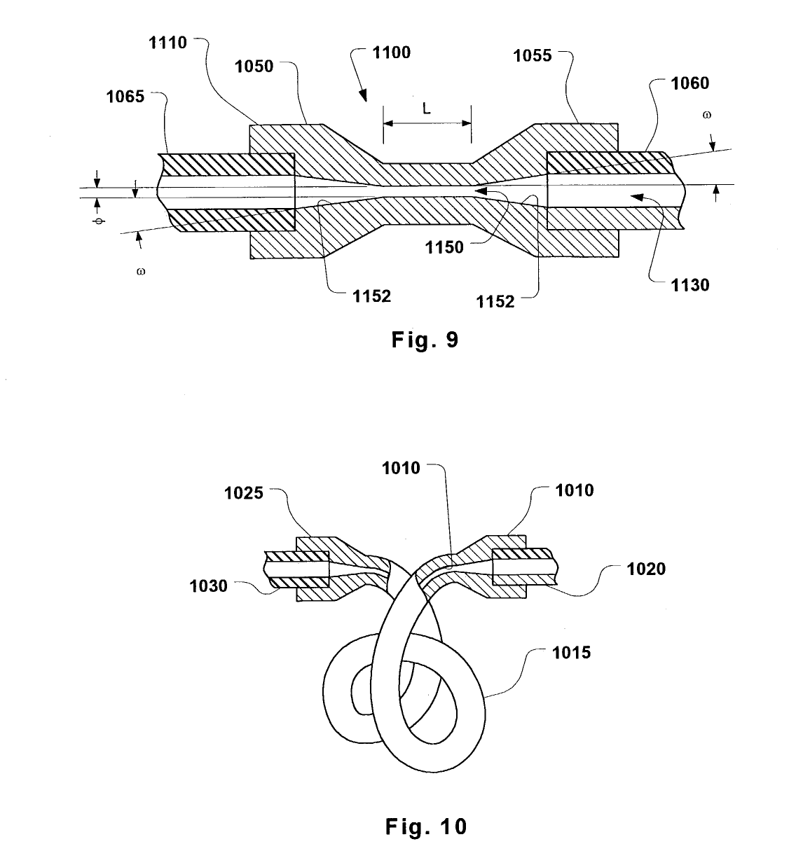

Referring now to FIG. 9, an alternative embodiment of a flow restrictor 1100 for generating the TMP desired for high membrane utilization has a molded portion 1110 with adapter portions 1050 and 1055 that receive tubing 1065 and 1060 at respective ends thereof. The molded portion 1110 has a flow channel 1130 that narrows progressively from the inner diameter of the tubing 1060 and 1065 to a smaller diameter portion 1150 that restricts the flow. A continuous flow channel 1130/1150 is thereby defined.

Preferably the profile 1152 defining the rate of decrease of the diameter of the flow channel 1130/1150, in the current embodiment, a simple conical angle indicated by ω, is such as to prevent laminar boundary layer separation. Alternatively, the profile may be selected to insure that any turbulence is at a low level determined to prevent more than a predefined amount of hemolysis. The above may be experimentally determined according to known techniques. In an embodiment, the angle ω may be set to 7° which in hemofiltration applications with blood flow rates in a typical range has proved adequate to limit hemolysis to tolerable levels.

Note that while a straight conical shape is represented above, it is clear that other shapes may also be used. For example, the contraction and expansions could have other profiles (e.g., curved) known in the field of fluid mechanics to minimize the potential for flow reversal and turbulence.

As an example, the restriction may be sized based on the following conditions: a hematocrit of 28 to 38 at a blood flow rate of 300-600 ml./min., the trans-membrane pressure (TMP) that corresponds to a filtrate rate of 33% of the blood flow rate and a waste pressure of at least 100 mm Hg. The diameter φ and the length L may be set to achieve the desired TMP at the given conditions.

Referring now to FIG. 10, a long flow restriction or capillary 1015 defines a restricted flow path 1010. Adapters 1025 and 1010 at either end permit connection to tubing 1030 and 1020, respectively. The long flow path provided by the capillary 1015 provides a higher pressure drop for a given flow acceleration/deceleration than the short flow restrictor shown at 1100 of FIG. 9. Again, the design parameters may be determined according to empirical design techniques as discussed above with respect to the FIG. 9 embodiment.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A blood treatment system, comprising:
a hemofilter having a blood inlet and an outlet and a filtrate outlet;
said outlet having a flow restrictor such that a significant pressure loss through the filter-flow restrictor combination occurs in the flow restrictor, whereby a high pressure differential is maintained between a portion of said filter nearest said flow restrictor and said filtrate outlet.

2. A system as in claim 1, further comprising a flow reversing mechanism which periodically and automatically reverses a flow of blood through said filter.

3. A system as in claim 1, wherein said flow restrictor is a capillary.

4. A method of hemofiltering the blood of a patient, comprising the steps of:
passing blood through a filter;
maintaining in said filter to a pressure differential between said blood and a filtrate side of said filter a pressure differential substantially greater than a pressure loss across a blood circuit through said filter such that a high utilization of media in said filter is achieved across an entire flow length thereof.

5. A method as in claim 4, wherein said step of maintaining includes subjecting a filtrate line of said filter to a vacuum.

6. A method as in claim 4, wherein said step of maintaining includes restricting a flow of blood at an outlet of said filter to increase a pressure of blood near said outlet and within said blood circuit through said filter.

7. A blood treatment system, comprising:
a hemofilter having a blood inlet and an outlet and a filtrate outlet;
said filtrate outlet having a vacuum pump connected thereto to maintain a vacuum on a filtrate side of said filter such that a pressure loss through said filter is substantially less than a pressure difference between a filtrate side of said filter and a blood side of said filter near said inlet of said filter.
